(19) United States
(12) Patent Application Publication
(10) Pub. No.: US 2019/0242908 A1
(43) Pub. Date: Aug. 8, 2019

(54) METHODS FOR DIAGNOSING AND TREATING NEPHROTIC SYNDROME

(71) Applicants: INSERM (INSTITUT NATIONAL DE LA SANTÉ ET DE LA RECHERCHE MÉDICALE), Paris (FR); ASSISTANCE PUBLIQUE-HÔPITAUX DE PARIS (APHP), Paris (FR); SORBONNE UNIVERSITE, Paris (FR)

(72) Inventor: Jean-Jacques BOFFA, Paris (FR)

(73) Assignees: INSERM (INSTITUT NATIONAL DE LA SANTÉ ET DE LA RECHERCHE MÉDICALE), Paris (FR); ASSISTANCE PUBLIQUE-HÔPITAUX DE PARIS (APHP), Paris (FR); SORBONNE UNIVERSITE, Paris (FR)

(21) Appl. No.: 16/331,662

(22) PCT Filed: Sep. 7, 2017
(86) PCT No.: PCT/EP2017/072492

§ 371 Date: Mar. 8, 2019

(30) Foreign Application Priority Data
Sep. 8, 2016 (EP) 16306124.5

(51) Int. Cl. G01N 33/68; C12Q 1/6883

(52) U.S. Cl. CPC G01N 33/6893; G01N 2800/347; C12Q 1/6883

(57) ABSTRACT

Inventors have shown that the protein isthmin-1 (ISM-1) is expressed at kidney level in animal and human model. They have also shown that this protein is expressed on the surface but also intracellular of the circulating leukocytes. They have observed that its expression is increased when a subject suffers from INS, MCN or FSGS compared to healthy controls. Among various causes of nephrotic syndrome, ISM-1 leucocyte expression is dramatically increased in patients with INS, MCN or FSGS. Accordingly, the present invention relates to a method for diagnosing INS, MCN or FSGS in a subject comprising the steps of: i) measuring the membrane expression level of isthmin-1 on the circulating leukocytes in a biological sample obtained from said subject; ii) comparing the expression level measured at step i) with its predetermined reference value, and iii) concluding that the subject suffers from INS, MCN or FSGS when the membrane expression level of isthmin-1 is higher than its predetermined reference value.

### FIELD OF THE INVENTION

The invention is in the field of nephrology, particularly, the invention relates to methods for diagnosing and treating nephrotic syndrome.

### BACKGROUND OF THE INVENTION

Nephrotic syndrome is a rare disease, defined by massive proteinuria (>3 g/day, or >1 g of urine protein per square meter of body-surface area per day in children) and hypoalbuminemia (<30 g/l) and result from loss of integrity of the glomerular filtration barrier. The main causes are genetic and immune. The diagnosis of the cause of nephrotic syndrome (NS) is based on a set of clinical, evolutionary, biochemical, therapeutic and pathological data. Idiopathic nephrotic syndrome (INS) represents 80% of the causes of NS in children and 25% in adults. INS is histologically characterized by the absence of lesion on light microscopy and absence of immunoglobulin or complement deposit with sometime additional focal segmental glomerusclerosis (FSGS) lesions. The first case defines the features of the minimal change nephropathy (MCN), the other FSGS. In current practice, to diagnose the precise cause of NS, a kidney biopsy is required. In adult, a kidney biopsy is systematically performed when a nephrotic syndrome is observed. In children, the diagnosis of INS is set up when nephrotic syndrome remission is observed after steroids treatment. MCN and FSGS are considered as a group of clinical-pathologic syndrome sharing a common glomerular lesion within the podocyte. On electron microscopy, flattening or effacement of foot processes represent the main lesions. Other immune causes of NS include membranous nephropathy, immune lupus nephritis, ANCA vasculitis, Goodpasture's disease, Berger's disease that could be easily diagnosed by their specific immune deposits and/or light micoscopic lesions. The pathophysiology of INS, at least in its primary form with its potential recurrence after transplantation remains unknown. It is admitted that INS has an immune origin with a putative circulating permeability factor. The occurrence of proteinuria is associated with a change in the adhesion of podocyte on its glomerular basement membrane, a rearrangement of the cytoskeleton and the slit diaphragm. A common signaling pathway initiates the disease process and leads to phosphorylation of tyrosine residues of nephrin and adhesion molecules such as VE-cadherin, β-catenin by different kinases Src, FAK, ILK. These phosphorylations lead to the dismantling of complex adhesion and membrane hyperpermeability. The main treatment is corticosteroids with diuretics (e.g furosemide or spironolactone). Treatment varies between adult and pediatric patients, kidney disease improving global outcomes (KDIGO) issued guidelines in 2012 that include recommendations on treatment of idiopathic nephrotic syndrome in adults and children. Unfortunately, some patients relapse and continue to suffer from INS and 10% will progress to end stage renal disease. Thus, there is a need to identify new markers which allow a better understanding of causation, new approaches to diagnosis and to predict the response to treatment and the relapse of patients.

### SUMMARY OF THE INVENTION

The present invention relates to a method for diagnosing idiopathic nephrotic syndrome (INS) in children, MCN or primitive FSGS in a subject comprising the steps of: i) measuring the membrane expression level of isthmin-1 on circulating leukocytes in a blood sample obtained from said subject; ii) comparing the expression measured at step i) with its predetermined reference value, and iii) concluding that the subject suffers from INS, MCN or primitive FSGS when the membrane expression level of isthmin-1 is higher than its predetermined reference value or concluding that the subject suffer from another cause of nephrotic syndrome when the membrane expression level of isthmin-1 is lower than its predetermined reference value. In particular, the present invention is defined by the claims.

### DETAILED DESCRIPTION OF THE INVENTION

For the first time, inventors have shown that the protein isthmin-1 (ISM-1) is physiologically expressed on podocytes, at kidney level in animal and human model. They have also shown that this protein is expressed on the surface but also intracellular of the circulating leukocytes. They have observed that its expression is increased when a subject suffers from all causes of nephrotic syndrome compared to healthy controls. Among various causes of nephrotic syndrome, ISM-1 leucocyte expression is dramatically increased in patients with INS, MCN, and primitive FSGS. Thus, the inventors have identified a new biomarker which is suitable to diagnosis INS, MCN or primitive FSGS from other causes of nephrotic syndrome, to predict the response to steroids treatment and to predict relapse of subject suffering from INS, MCN or FSGS.

Method for Diagnosing Nephrotic Syndrome

Accordingly, in a first aspect, the invention relates to a method for diagnosing idiopathic nephrotic syndrome (INS), minimal change nephropathy (MCN) or primitive Focal Segmental Glomerulosclerosis (FSGS) in a subject comprising the steps of: i) measuring the membrane expression level of isthmin-1 on the circulating leukocytes in a biological sample obtained from said subject; ii) comparing the expression level measured at step i) with its predetermined reference value, and iii) concluding that the subject suffers from INS, MCN or FSGS when the membrane expression level of isthmin-1 is higher than its predetermined reference value or concluding that the subject does not suffer from INS, MCN or FSGS when the membrane expression level of isthmin-1 is lower or similar than its predetermined reference value.

As used herein term “diagnosing” refers to classifying a disease or a symptom, determining a severity of the disease, monitoring disease progression, forecasting an outcome of a disease and/or prospects of recovery.

As used herein, the term “idiopathic nephrotic syndrome” (INS) has its general meaning in the art and represents 80% of the causes of nephrotic syndrome (NS) in children and 25% in adults. INS is histologically characterized by the absence of lesion on light microscopy and absence of immunoglobulin or complement deposit with sometime additional focal segmental glomerusclerosis (FSGS) lesions. As used herein, the term “MCN”, also known as minimal change disease (MCD), lipoid nephrosis or nil disease, refers to minimal change nephropathy. It arises from a histopathologic lesion in the glomerulus and is characterized by intense proteinuria leading to edema and intravascular volume depletion. It is the most common single form of nephrotic syndrome in children, but it can also occur in adults. In a particular embodiment, the nephrotic syndrome is caused by idiopathic nephrotic syndrome (INS). As used herein, the term “FSGS” refers to a rare disease that attacks the kidney's filtering units (glomeruli) causing serious scarring which leads to permanent kidney damage and even failure. The term “focal” is added because in FSGS, only some of the glomeruli filters become scarred. The term “Segmental” means that only some sections of the glomerulus becomes scarred, just parts of them.

In a particular embodiment, INS, MCN or FSFS cause the nephrotic syndrome. As used herein, the term “nephrotic syndrome” is a kidney disorder that causes the body to excrete too much protein in the urine. Nephrotic syndrome has many causes, including primary kidney diseases such as minimal change nephropathy, focal glomerulosclerosis, and membranous nephropathy . . . . Nephrotic syndrome is characterized by large proteinuria, hypoalbuminemia, hyperlipidaemia, and edema.

As used herein, the term “subject” refers to any mammals, such as a rodent, a feline, a canine, and a primate. Particularly, in the present invention, the subject is a human. In a particular embodiment, the subject is a human who is susceptible to have nephrotic syndrome. More particularly, the subject is susceptible to have INS, MCN or FSGS which cause nephrotic syndrome.

As used herein, the term “isthmin-1” or ISM-1 refers to a secreted protein identified firstly in Xenopus. It is highly expressed in the isthmus of the midbrain—hindbrain organizer in Xenopus with unknown functions. The gene ISM-1 encodes a secreted 60 kD protein containing a thrombospondin type 1 (TSR1) repeat domain in the central region and an adhesion-associated domain in MUC4 and other proteins (AMOP) domain at the C-terminal.

The naturally occurring human isthmin gene has a nucleotide sequence as shown in Genbank Accession number NM 080826 and the naturally occurring human isthmin protein has an aminoacid sequence as shown in Genbank Accession number NP_543016.1. The murine nucleotide and amino acid sequences have also been described (Genbank Accession numbers NM 001276489 and NP_001263418.1).

As used herein, the term “circulating leukocytes”, also called white blood cells (WBCs) or leucocytes refers to the cells of the immune system that are involved in protecting the body against infections and all foreign invaders. The term “leukocytes” encompasses neutrophils, monocytes/macrophages, lymphocytes, and mixtures thereof. The term “circulating” refers to the naturally-occurring cells which are “circulating” in the bloodstream of the subject, which means they have not been injected into the subject, removed from the subject, cultured, and/or re-injected into the subject. In some embodiments, the circulating leukocytes are neutrophil, eosinophil, basophil, lymphocytes (e.g B cells, T cells or NK cells) or monocytes (e.g macrophages). Circulating leukocytes are obtained from a biological sample.

As used herein, the term “biological sample” refers to a sample obtained from a subject, for example blood, saliva, breast milk, urine, semen, blood plasma, synovial fluid or serum. In a particular embodiment, the sample is blood sample. The term “blood sample” means any blood sample derived from the subject. Peripheral blood is preferred, and mononuclear cells (PBMCs) are the preferred cells. Typically, these cells can be extracted from whole blood using Ficoll, a hydrophilic polysaccharide that separates layers of blood, with the PBMC forming a cell ring under a layer of plasma. Additionally, PBMC can be extracted from whole blood using a hypotonic lysis which will preferentially lyse red blood cells. Such procedures are known to the expert in the art.

As used herein, the term “measuring membrane expression level” refers to quantify the expression level on the cell surface. Methods for determining or measuring the expression level of a marker on the surface cell is are well known in the art. The detection and quantification of a marker that is expressed by a cell is performed by flow cytometry. In some embodiments, such method comprises contacting the sample with at least one selective binding agent capable of selectively interacting with the protein of interest (i.e. isthmin-1). The selective binding agent may be polyclonal antibody or monoclonal antibody, an antibody fragment, synthetic antibodies, or other protein-specific agents such as nucleic acid or peptide aptamers. For the detection of the antibody that makes the presence of the marker detectable by microscopy or an automated analysis system, the antibodies may be tagged directly with detectable labels such as enzymes, chromogens or fluorescent probes or indirectly detected with a secondary antibody conjugated with detectable labels. The binding agents such as antibodies or aptamers may be labelled with a detectable molecule or substance, such as preferentially a fluorescent molecule, or a radioactive molecule or any others labels known in the art. As used herein, the terms “label” and “detectable label” refer to a molecule capable of detection, including, but not limited to, radioactive isotopes, fluorescers, chemiluminescers, chromophores, enzymes, enzyme substrates, enzyme cofactors, enzyme inhibitors, chromophores, dyes, metal ions, metal sols, ligands (e.g., biotin, avidin, streptavidin or haptens), intercalating dyes and the like. The term “fluorescer” refers to a substance or a portion thereof which is capable of exhibiting fluorescence in the detectable range. Labels of interest include both directly and indirectly detectable labels. Suitable labels for use in the methods described herein include any molecule that is indirectly or directly detectable by spectroscopic, photochemical, biochemical, immunochemical, electrical, optical, chemical, or other means. Labels of interest include, but are not limited to, fluorescein and its derivatives; rhodamine and its derivatives; cyanine and its derivatives; coumarin and its derivatives; Cascade Blue and its derivatives; Lucifer Yellow and its derivatives; BODIPY and its derivatives; and the like. Labels of interest also include fluorophores, such as indocarbocyanine (C3), indodicarbocyanine (C5), Cy3, Cy3.5, Cy5, Cy5.5, Cy7, Texas Red, Pacific Blue, Oregon Green 488, Alexa fluor-355, Alexa Fluor 488, Alexa Fluor 532, Alexa Fluor 546, Alexa Fluor-555, Alexa Fluor 568, Alexa Fluor 594, Alexa Fluor 647, Alexa Fluor 660, Alexa Fluor 680, Alexa Fluor 700, JOE, Lissamine, Rhodamine Green, BODIPY, fluorescein isothiocyanate (FITC), carboxy-fluorescein (FAM), phycoerythrin, rhodamine, dichlororhodamine (dRhodamine), carboxy tetramethylrhodamine (TAMRA), carboxy-X-rhodamine (ROX), LIZ, VIC, NED, PET, SYBR, PicoGreen, RiboGreen, and the like. Fluorescent labels can be detected using a photodetector (e.g., in a flow cytometer) to detect emitted light. Enzymatic labels are typically detected by providing the enzyme with a substrate and detecting the reaction product produced by the action of the enzyme on the substrate, colorimetric labels can be detected by simply visualizing the colored label, and antigenic labels can be detected by providing an antibody (or a binding fragment thereof) that specifically binds to the antigenic label. An antibody that specifically binds to an antigenic label can be directly or indirectly detectable. For example, the antibody can be conjugated to a label moiety (e.g., a fluorophore) that provides the signal (e.g., fluorescence); the antibody can be conjugated to an enzyme (e.g., peroxidase, alkaline phosphatase, etc.) that produces a detectable product (e.g., fluorescent product) when provided with an appropriate substrate (e.g., fluorescent-tyramide, FastRed, etc.); etc. The aforementioned assays may involve the binding of the binding agents (ie. antibodies or aptamers) to a solid support. The solid surface could a microtitration plate coated with the binding partner. Alternatively, the solid surfaces may be beads, such as activated beads, magnetically responsive beads. Beads may be made of different materials, including but not limited to glass, plastic, polystyrene, and acrylic. In addition, the beads are preferably fluorescently labelled. In a preferred embodiment, fluorescent beads are those contained in TruCount™ tubes, available from Becton Dickinson Biosciences, (San Jose, Calif.). According to the invention, methods of flow cytometry are preferred methods for measuring the level of the protein of interest (i.e.isthmin-1). Flow cytometry is a well-accepted tool in research that allows a user to rapidly analyze and sort components in a sample fluid. Flow cytometers use a carrier fluid (e.g., a sheath fluid) to pass the sample components, substantially one at a time, through a zone of illumination. Each sample component is illuminated by a light source, such as a laser, and light scattered by each sample component is detected and analyzed. The sample components can be separated based on their optical and other characteristics as they exit the zone of illumination. Said methods are well known in the art. In a particular embodiment, a fluorescence activated cell sorting (FACS) is used to measure the expression level of ISM-1. Involves using a flow cytometer capable of simultaneous excitation and detection of multiple fluorophores, such as a BD Biosciences FACSCanto™ flow cytometer, used substantially according to the manufacturer's instructions. The cytometric systems may include a cytometric sample fluidic subsystem, as described below. In addition, the cytometric systems include a cytometer fluidically coupled to the cytometric sample fluidic subsystem. Systems of the present disclosure may include a number of additional components, such as data output devices, e.g., monitors, printers, and/or speakers, data input devices, e.g., interface ports, a mouse, a keyboard, etc., fluid handling components, power sources, etc.

In another embodiment, the measure of the ISM-1 expression level is carried out by immunological detection. Typically, the immunological detection or quantification of the ISM-1 expression level is achieved by any methods known in the art using at least one antibody that binds specifically to ISM-1. Examples of said methods include, but are not limited to, standard electrophoretic and immunodiagnostic techniques such as western blots, immuno-precipitation assay, radioimmunoassay, ELISA (enzyme-linked immunosorbent assay), “sandwich” immunoassay, gel diffusion precipitation reaction, immunodiffusion assay, precipitation reaction, agglutination assay (such as gel agglutination assay, hemagglutination assay, etc.), complement fixation assay, protein A assay, immunoelectrophoresis assay, high performance liquid chromatography, size exclusion chromatography, solid-phase affinity, etc. In a particular embodiment, the expression level of ISM-1 is measured by ELISA.

As used herein, the term “predetermined reference value” refers to a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement in properly banked historical subject samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after determining the expression level of the selected peptide in a group of reference, one can use algorithmic analysis for the statistic treatment of the expression levels determined in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

The inventors have shown that isthmin-1 has also an intracellular expression in the circulating leukocytes. They have established that the ratio of the intracellular expression of isthmin-1 to the membrane expression of isthmin-1 is suitable to diagnosis a subject who is susceptible to have idiopathic nephrotic syndrome (INS), minimal change nephropathy (MCN) or Focal Segmental Glomerulosclerosis (FSGS). Thus, the invention relates to diagnose these diseases which cause the nephropathy syndrome.

Accordingly, in a second aspect, the present invention relates to a method for diagnosing idiopathic nephrotic syndrome (INS), minimal change nephropathy (MCN) or Focal Segmental Glomerulosclerosis (FSGS) in a subject comprising the steps of: i) measuring the intracellular expression level of isthmin-1 in the circulating leukocytes in a sample obtained from said subject; ii) measuring the membrane expression level of isthmin-1 on the circulating leukocytes in said sample, iii) calculating the ratio of the intracellular expression level of isthmin-1 determined at step i) to the membrane expression level of isthmin-1 determined at step ii); iv) comparing the ratio determined at step iii) with a predetermined reference value, and v) concluding that the subject suffers from idiopathic nephrotic syndrome (INS), minimal change nephropathy (MCN) or Focal Segmental Glomerulosclerosis (FSGS) when the ratio determined at step iii) is higher than the predetermined reference value or concluding that the subject does not suffer from nephrotic syndrome when the ratio determined at step iv) is lower than the predetermined reference value.

As used herein, the term “measuring intracellular expression level” refers to determinate or quantify the expression or presence of a marker in the cell. Methods for determining or measuring the expression of a marker in the cell is are well known in the art. The detection and quantification of a marker that is expressed by a cell is performed by flow cytometry. As being also intra cellularly located, isthmin expression may be assessed by intracellular flow cytometry using a labeled anti isthmin-1 antibodies. Intracellular flow cytometry typically involves the permeabilization and fixation of the cells (e.g. T cells). Any convenient means of permeabilizing and fixing the cells may be used in practicing the methods. For example permeabilizing agent typically include saponin, methanol, Tween® 20, Triton X-100™. In a particular embodiment, intracellular expression level of ISM-1 is performed by immunological detection as described above.

Method for Diagnosing Dysfunction of Capillary Permeability

Inventors have shown that isthmin-1 has a role in the vascular permeability, particularly, they have demonstrated that isthmin-1 increases the capillary permeability. Thus, ISM-1 is suitable to predict or diagnose a subject suffering from a vascular permeability dysfunction.

Accordingly, in another aspect, the invention relates to a method for diagnosing dysfunction of capillary permeability in a subject comprising the steps of: i) measuring the membrane expression level of isthmin-1 on the circulating leukocytes in a biological sample obtained from said subject; ii) comparing the expression level measured at step i) with its predetermined reference value, and iii) concluding that the subject suffers from dysfunction of capillary permeability when the membrane expression level of isthmin-1 is higher than its predetermined reference value or concluding that the subject does not suffer from dysfunction of capillary permeability when the membrane expression level of isthmin-1 is lower or similar than its predetermined reference value.

As used herein, the term “capillary permeability” refers to the capacity of a blood vessel wall to allow for the flow of small molecules (drugs, nutrients, water, ions) or even whole cells (lymphocytes on their way to the site of inflammation) in and out of the vessel. In a particular embodiment, the capillary permeability refers to the glomerular capillary permeability. The term “glomerular capillary permeability” in a healthy subject, refers to the capillary endothelium of the glomerulus, by virtue of its fenestration, is permeable to all blood constituents except blood cells and colloids so that the glomerular filtrate has a close similarity to plasma and interstitial fluid but has a lower protein concentration than both of them. The terms “dysfunction of glomerular capillary permeability” or “impaired glomerular capillary permeability” refer to various abnormalities, including, for example, disturbance or impairment of the structure and/or function of the glomerular vasculature. An increase of vascular permeability is one of the main characteristics of the inflammatory response of the body against stimuli, especially in the case of acute inflammation. During inflammation, the chemical factors derived from plasma and triggered by inflammatory stimuli mediate a number of vascular and cellular responses in the affected site. These structural changes in the microvasculature result in increased permeability of the blood vessel membrane, causing movement of plasma proteins and cells, e.g. leukocytes from the circulation to the intersititium. The main mechanisms of increased vascular permeability in inflammation include endothelial cell contraction, junctional retraction, direct injury, leukocyte-dependent leakage, regenerating endothelium, amongst others. Increased fluid filtration towards the interstitium is further enhanced by the arteriolar vasodilator action of the inflammatory mediators, which increases the blood flow, the perfused surface area and capillary hydrostatic pressure.

In a particular embodiment, the invention relates to a method of diagnosing dysfunction of glomerular capillary permeability in a subject comprising the steps of: i) measuring the intracellular expression level of isthmin-1 in the circulating leukocytes in a sample obtained from said subject; ii) measuring the membrane expression level of isthmin-1 on the circulating leukocytes in said sample, iii) calculating the ratio of the intracellular expression level of isthmin-1 determined at step i) to the membrane expression level of isthmin-1 determined at step ii); iv) comparing the ratio determined at step iii) with a predetermined reference value, and v) concluding that the subject suffers from dysfunction of glomerular capillary permeability when the ratio determined at step iii) is higher than the predetermined reference value or concluding that the subject does not suffer from dysfunction of glomerular capillary permeability when the ratio determined at step iv) is lower than the predetermined reference value.

Method for Predicting the Response to Steroids and the Risk of Relapse

Data from inventor show that patients with idiopathic nephrotic syndrome (INS), minimal change disease (MCD) or primitive Focal Segmental Glomerulosclerosis (FSGS) and with high level leucocyte expression of Isthmin-1 have a very good response to steroids treatment. All of them had a complete remission of idiopathic nephrotic syndrome (INS), minimal change disease (MCD) or Focal Segmental Glomerulosclerosis (FSGS) in a few weeks contrasting with patients without low Isthmin-1 expression. Accordingly, the invention relates to diagnose these diseases which cause the nephropathy syndrome.

Accordingly, in a third aspect, the invention relates to a method for predicting the risk of relapse to treatment in a subject suffering from idiopathic nephrotic syndrome (INS), minimal change nephropathy (MCN) or Focal Segmental Glomerulosclerosis (FSGS) comprising the steps of: i) measuring the membrane expression level of isthmin-1 on the circulating leukocytes in a sample obtained from said subject; ii) comparing the expression level measured at step i) with its predetermined reference value, and iii) concluding that the subject is at risk of relapse to the treatment when the membrane expression level of isthmin-1 is higher than its predetermined reference value or concluding that the subject is not at risk of relapse when the membrane expression level of isthmin-1 is lower than its predetermined reference value.

As used herein, the term “predicting” means that the subject to be analyzed by the method of the invention is allocated either into the group of subjects who will relapse, or into a group of subjects who will not relapse after a treatment.

As used herein, the term “risk” in the context of the present invention, relates to the probability that an event will occur over a specific time period, as in the conversion to relapse, and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event) to no-conversion. “Risk evaluation,” or “evaluation of risk” in the context of the present invention encompasses making a prediction of the probability, odds, or likelihood that an event or disease state may occur, the rate of occurrence of the event or conversion from one disease state to another, i.e., from a normal condition to relapse or to one at risk of developing relapse. Risk evaluation can also comprise prediction of future clinical parameters, traditional laboratory risk factor values, or other indices of relapse, either in absolute or relative terms in reference to a previously measured population. The methods of the present invention may be used to make continuous or categorical measurements of the risk of conversion to relapse, thus diagnosing and defining the risk spectrum of a category of subjects defined as being at risk of having relapse. In the categorical scenario, the invention can be used to discriminate between normal and other subject cohorts at higher risk of having relapse. In some embodiments, the present invention may be used so as to discriminate those at risk of having relapse from normal, or those having relapse disease from normal.

As used herein, the term “relapse” refers to the return of signs and symptoms of a disease after a subject has enjoyed a remission after a treatment. Thus, if initially the target disease is alleviated or healed, or progression of the disease was halted or slowed down, and subsequently the disease or one or more characteristics of the disease return, the subject is referred to as being “relapsed.”

In some embodiments, the method of the present invention is particularly suitable for predicting the risk of relapse when the subject was or is treated with a least one agent selected from the group consisting of immunosuppressive drugs and corticosteroids. As used herein, the term “immunosuppressive drug” refers to any substance capable of producing an immunosuppressive effect, e.g., the prevention or diminution of the immune response.

Immunosuppressive drugs include, without limitation thiopurine drugs such as azathioprine (AZA) and metabolites thereof nucleoside triphosphate inhibitors such as mycophenolic acid (Cellcept) and its derivative (Myfortic); derivatives thereof prodrugs thereof and combinations thereof. In some embodiments the immunosuppressive drug is ciclosporin (also named “ciclosporin” A or “CyA”) that is a competitive calcineurin inhibitor with potent immunosuppressive properties.

As used, the term “corticosteroids” has its general meaning in the art and refers to class of active ingredients having a hydrogenated cyclopentoperhydrophenanthrene ring system endowed with an anti-inflammatory activity. Corticosteroid drugs typically include cortisone, cortisol, hydrocortisone (11β,17-dihydroxy, 21-(phosphonooxy)-pregn-4-ene, 3,20-dione disodium), dihydroxycortisone, dexamethasone (21-(acetyloxy)-9-fluoro-1β,17-dihydroxy-16α-m-ethylpregna-1,4-diene-3,20-dione), and highly derivatized steroid drugs such as beconase (beclomethasone dipropionate, which is 9-chloro-11-β, 17,21, trihydroxy-16β-methylpregna-1,4 diene-3,20-dione 17,21-dipropionate). Other examples of corticosteroids include flunisolide, prednisone, prednisolone, methylprednisolone, triamcinolone, deflazacort and betamethasone. corticosteroids, for example, cortisone, hydrocortisone, methylprednisolone, prednisone, prednisolone, betamethesone, beclomethasone dipropionate, budesonide, dexamethasone sodium phosphate, flunisolide, fluticasone propionate, triamcinolone acetonide, betamethasone, fluocinolone, fluocinonide, betamethasone dipropionate, betamethasone valerate, desonide, desoximetasone, fluocinolone, triamcinolone, triamcinolone acetonide, clobetasol propionate, and dexamethasone.

In a fourth aspect, the present invention relates to a method for predicting the risk of relapse to a treatment in a subject suffering from idiopathic nephrotic syndrome (INS), minimal change nephropathy (MCN) or Focal Segmental Glomerulosclerosis (FSGS) comprising the steps of: i) measuring the intracellular expression level of isthmin-1 in the circulating leukocytes in a sample obtained from said subject; ii) measuring the membrane expression level of isthmin-1 on the circulating leukocytes in said sample; iii) calculating the ratio of the expression level determined at step i) to the expression level determined at step ii); iv) comparing the ratio determined at step iii) with a predetermined reference value, and v) concluding that the subject is at risk of relapse when the ratio determined at step iii) is higher than its predetermined reference value or concluding that the subject is not at risk of relapse when the ratio determined at step iii) is lower than its predetermined reference value.

In some embodiments, it is concluded that the subject is not at risk of relapse (e.g. remission) when the expression of isthmin-1 on circulating leukocytes is the lower than its predetermined reference value.

Method for Determining whether a Renal Biopsy is Required or not in a Subject

The method of the present invention is particularly suitable for determining whether a renal biopsy is required in a subject.

Typically, when the diagnosis method as described above concludes that the subject does not suffer from idiopathic nephrotic syndrome (INS), minimal change nephropathy (MCN) or Focal Segmental Glomerulosclerosis (FSGS), the physician can decide performing a renal biopsy to clarify the diagnosis.

Renal biopsy exposes the subjects to severe complications such as severe hematuria, hemorrhage, arterial injury, transfusion, requiring sometimes arterial embolization, nephrectomy, and prolonged hospitalization. In children, performing renal biopsy is often difficult. So the method of the invention offers a mean to avoid the renal biopsy if it is not necessary. Indeed, when it is concluded that the diagnosis of INS is likely based on flow cytometer data or ELISA the physician can decide to avoid renal biopsy. In the opposite side, when this test is not in favor of INS disease, the physician can decide performing a renal biopsy to clarify the diagnosis. Consequently, the method of the present invention would provide a new classification of ISN, MCN and primitive FSGS depending of ISM-1 expression.

Method for Monitoring the Progress of Nephrotic Syndrome

In some embodiments, the invention relates to a method for monitoring the progress of idiopathic nephrotic syndrome (INS), minimal change disease (MCD) or Focal Segmental Glomerulosclerosis (FSGS) in a subject wherein, a first measuring ishtmin-1 on circulating leukocytes in a sample obtained from said subject is performed during the course of the treatment and a second measurement of ishtmin-1 in sample is performed later (after several hours, days or months) and concluding that the subject would be at high relapse risk when the expression of isthmin-1 increases between the two measurements.

The method of the present invention is thus particularly suitable for adjusting the treatment of the subject e.g. by adjusting the dosage, combining with administration of a new drug, substituting the current treatment by a new one.

Kit for Diagnosis INS or for Predict the Risk of Relapse

In a fifth aspect, the present invention relates to a kit comprising means for performing the method of the present invention. Typically, the kit comprises means for detecting expression of isthmin-1. In some embodiments, the means are antibodies labelled. Typically, the kits described above will also comprise one or more other containers, containing for example, wash reagents, and/or other reagents capable of quantitatively detecting the presence of bound antibodies. The kit also contains agents suitable for performing flow cytometry or ELISA. Typically compartmentalised kit includes any kit in which reagents are contained in separate containers, and may include small glass containers, plastic containers or strips of plastic or paper. Such containers may allow the efficient transfer of reagents from one compartment to another compartment whilst avoiding cross-contamination of the samples and reagents, and the addition of agents or solutions of each container from one compartment to another in a quantitative fashion. Such kits may also include a container which will accept the blood sample, a container which contains the antibody(s) used in the assay, containers which contain wash reagents (such as phosphate buffered saline, Tris-buffers, and like), and containers which contain the detection reagent.

Method and Compositions for Treating Nephrotic Syndrome

In a sixth aspect, the invention relates to a method of treating idiopathic nephrotic syndrome (INS), minimal change nephropathy (MCN) or Focal Segmental Glomerulosclerosis (FSGS) in a subject in need thereof comprising a step of administering to the subject a therapeutically effective amount of inhibitors of isthmin-1.

More particularly, the method of the invention is suitable to treat nephrotic syndrome which is caused by idiopathic nephrotic syndrome (INS), minimal change nephropathy (MCN) or Focal Segmental Glomerulosclerosis (FSGS).

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “subject” refers to any mammals, such as a rodent, a feline, a canine, and a primate. Particularly, in the present invention, the subject is a human. In a particular embodiment, the subject is a human who is susceptible to have nephrotic syndrome idiopathic nephrotic syndrome. More particularly, the subject is susceptible to have INS, MCN or FSGS which cause nephrotic syndrome. As used herein, the term “inhibitors of isthmin-1” refers to a natural or synthetic compound that has a biological effect to inhibit or significantly reduce the activity or expression of the transcripts and/or proteins. Thus, an “inhibitor of ISM-1” refers to a natural or synthetic compound that has a biological effect to inhibit or significantly reduce the activity or expression of ISM-1 transcripts and/or proteins. In a particular embodiment, the inhibitor of ISM-1 is an inhibitor of ISM-1 activity. The term “inhibitor of ISM-1 activity” has its general meaning in the art, and refers to a compound which has the capability of reducing or suppressing selectively the activity of ISM-1. Typically, an inhibitor of ISM-1 activity is a small organic molecule, a polypeptide, an aptamer or an antibody.

In some embodiments, the inhibitor of isthmin-1 activity is a small organic molecule.

The term “small organic molecule” as used herein, refers to a molecule of a size comparable to those organic molecules generally used in pharmaceuticals. The term excludes biological macro molecules (e. g. proteins, nucleic acids, etc.). Typically, small organic molecules range in size up to about 5000 Da, more preferably up to 2000 Da, and most preferably up to about 1000 Da.

In some embodiments, the inhibitor of isthmin-1 activity is an antibody. More particularly, the antibody is suitable to inhibit ISM-1 present on the leukocytes membrane. The term “antibody” is thus used to refer to any antibody-like molecule that has an antigen binding region, and this term includes antibody fragments that comprise an antigen binding domain such as Fab′, Fab, F(ab′)2, single domain antibodies (DABs or VHH), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art. In some embodiments, the antibody is a monoclonal antibody. In some embodiments, the antibody is non-internalizing. As used herein the term “non-internalizing antibody” refers to an antibody, respectively, that has the property of to bind to a target antigen present on a cell surface, and that, when bound to its target antigen, does not enter the cell and become degraded in the lysosome. Particularly, in the context of the invention, the antibody is a single domain antibody. The term “single domain antibody” has its general meaning in the art and refers to the single heavy chain variable domain of antibodies of the type that can be found in Camelid mammals which are naturally devoid of light chains. Such single domain antibody are also called VHH or “Nanobody®”. For a general description of (single) domain antibodies, reference is also made to the prior art cited above, as well as to EP 0 368 684, Ward et al. (Nature 1989 Oct. 12; 341 (6242): 544-6), Holt et al., Trends Biotechnol., 2003, 21(11):484-490; and WO 06/030220, WO 06/003388. In the context of the invention, the amino acid residues of the single domain antibody are numbered according to the general numbering for VH domains given by the International ImMunoGeneTics information system aminoacid numbering (http://imgt.cines.fr/). Particularly, in the context of the invention, the antibody is a single chain variable fragment. The term “single chain variable fragment” or “scFv fragment” refers to a single folded polypeptide comprising the VH and VL domains of an antibody linked through a linker molecule. In such a scFv fragment, the VH and VL domains can be either in the VH-linker-VL or VL-linker-VH order. In addition to facilitate its production, a scFv fragment may contain a tag molecule linked to the scFv via a spacer. A scFv fragment thus comprises the VH and VL domains implicated into antigen recognizing but not the immunogenic constant domains of corresponding antibody.

In some embodiments, the inhibitor of isthmin-1 activity is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity.

In some embodiments, the inhibitor of isthmin-1 activity is a polypeptide. The term “polypeptide” refers to a polypeptide that specifically bind to ISM-1, can be used as an ISM-1 inhibitor that bind to and sequester the ISM-1 protein, thereby preventing it from signaling. Polypeptide refers both short peptides with a length of at least two amino acid residues and at most 10 amino acid residues, oligopeptides (11-100 amino acid residues), and longer peptides (the usual interpretation of “polypeptide”, i.e. more than 100 amino acid residues in length) as well as proteins (the functional entity comprising at least one peptide, oligopeptide, or polypeptide which may be chemically modified by being glycosylated, by being lipidated, or by comprising prosthetic groups). The definition of polypeptides also comprises native forms of peptides/proteins in mycobacteria as well as recombinant proteins or peptides in any type of expression vectors transforming any kind of host, and also chemically synthesized peptides.

In a particular embodiment, the inhibitor of isthmin-1 is an inhibitor of isthmin-1 expression. An “inhibitor of isthmin-1 expression” refers to a natural or synthetic compound that has a biological effect to inhibit or significantly reduce the expression of the gene encoding for ISM-1. Typically, the inhibitor of isthmin-1 expression has a biological effect on one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

In some embodiments, the inhibitor of isthmin-1 expression is an antisense oligonucleotide. Anti-sense oligonucleotides, including anti-sense RNA molecules and anti-sense DNA molecules, would act to directly block the translation of ISM-1 mRNA by binding thereto and thus preventing protein translation or increasing mRNA degradation, thus decreasing the level of ISM-1 proteins, and thus activity, in a cell. For example, antisense oligonucleotides of at least about 15 bases and complementary to unique regions of the mRNA transcript sequence encoding ISM-1 can be synthesized, e.g., by conventional phosphodiester techniques and administered by e.g., intravenous injection or infusion. Methods for using antisense techniques for specifically alleviating gene expression of genes whose sequence is known are well known in the art (e.g. see U.S. Pat. Nos. 6,566,135; 6,566,131; 6,365,354; 6,410,323; 6,107,091; 6,046,321; and 5,981,732).

In some embodiments, the inhibitor of isthmin-1 expression is a small inhibitory RNAs (siRNAs). ISM-1 expression can be reduced by contacting the subject or cell with a small double stranded RNA (dsRNA), or a vector or construct causing the production of a small double stranded RNA, such that ISM-1 expression is specifically inhibited (i.e. RNA interference or RNAi). Methods for selecting an appropriate dsRNA or dsRNA-encoding vector are well known in the art for genes whose sequence is known (e.g. see Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

In some embodiments, inhibitor of isthmin-1 expression is a ribozyme. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of ISM-1 mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

In some embodiments, the inhibitor of isthmin-1 expression is an endonuclease. The term “endonuclease” refers to enzymes that cleave the phosphodiester bond within a polynucleotide chain. Some, such as Deoxyribonuclease I, cut DNA relatively nonspecifically (without regard to sequence), while many, typically called restriction endonucleases or restriction enzymes, and cleave only at very specific nucleotide sequences. The mechanism behind endonuclease-based genome inactivating generally requires a first step of DNA single or double strand break, which can then trigger two distinct cellular mechanisms for DNA repair, which can be exploited for DNA inactivating: the errorprone nonhomologous end-joining (NHEJ) and the high-fidelity homology-directed repair (HDR). In a particular embodiment, the endonuclease is CRISPR-cas. As used herein, the term “CRISPR-cas” has its general meaning in the art and refers to clustered regularly interspaced short palindromic repeats associated which are the segments of prokaryotic DNA containing short repetitions of base sequences. In some embodiment, the endonuclease is CRISPR-cas9 which is from Streptococcus pyogenes. The CRISPR/Cas9 system has been described in U.S. Pat. No. 8,697,359 B1 and US 2014/0068797. In some embodiment, the endonuclease is CRISPR-Cpf1 which is the more recently characterized CRISPR from Provotella and Francisella 1 (Cpf1) in Zetsche et al. (“Cpf1 is a Single RNA-guided Endonuclease of a Class 2 CRISPR-Cas System (2015); Cell; 163, 1-13).

In a particular embodiment, the inhibitor of isthmin-1 is an inhibitor of isthmin-1 receptor. As known in the art, the receptor of ISM-1 is αvβ5 integrin. The term “αvβ5 integrin” refers to integrin alpha V and integrin beta 5. αvβ5 integrin is a member of a family of adhesion molecules that comprise non-covalently associated α/β heterodimers that mediate, inter alia, cell-cell interactions, cell-extracellular matrix (ECM) interactions, and cell-pathogen interactions. αvβ5 is the only integrin that contains the 05 subunit. αvβ5 recognizes the RGD peptide sequence and binds vitronectin (see, e.g., Hynes, Cell 69:11-25 (1992) and has been implicated in multiple disorders including stroke, myocardial infarction, cancer (i.e., angiogenesis), and ocular neovascularization disease. In some embodiments, the inhibitor of ISM-1 is an inhibitor of αvβ5 integrin. Typically, the inhibitor of αvβ5 integrin is any compound that competes with a αvβ5 ligand for available ligand binding sites on αvβ5 integrin. In the context of the invention, the ligand is ISM-1. Accordingly, the inhibitor of αvβ5 integrin prevents the interaction between αvβ5 integrin and ISM-1. αvβ5 integrin inhibitors include compounds that specifically bind to αvβ5 or β5, or that can inhibit the activity or expression of αvβ5 integrin. Examples include antibodies, small molecule inhibitors, antisense oligonucleotides or siRNA. Typically, the inhibitor of αvβ5 integrin is a small molecule. In a particular embodiment, the small molecule is Tyrosine alkoxyguanidines as described in WO2000047552. In a particular embodiment, the small molecule is tricyclic indanyls as described in U.S. Pat. No. 7,351,711. In a particular embodiment, the small molecule is a compound as described in WO2011098603. In a particular embodiment, the small molecule is biphenyl and its derivatives as described in WO0216323. In a particular embodiment, the small molecule is cilengitide (EMD 121974) as described in WO 0015244. In a particular embodiment, the inhibitor of αvβ5 integrin is an antibody. In a particular embodiment, the antibody is a monoclonal antibody. In a particular embodiment, the monoclonal antibody is mAb 17E6 (EMD 73034) as described in EP 719859. In a particular embodiment, the monoclonal antibody is a recombinant anti-av-integrin hybrid antibody as described in WO2009010290. In a particular embodiment, the monoclonal antibody is CNTO 95 as described in Shoucheng Ning et al 2008.

In a particular embodiment, the inhibitor of isthmin-1 is an inhibitor of GRP-78 (BiP). As known in the art, the receptor of ISM-1 is also GRP-78. As used, herein, the term “GRP-78”, also known as binding immunoglobulin protein (BiP), 78 kDa glucose-regulated protein (GRP-78) or heat shock 70 kDa protein 5 (HSPAS) is a protein that in humans is encoded by the HSPAS gene. GRP-78 plays a central role in regulating the unfolded protein response (UPR), and is an obligatory component of autophagy in mammalian cells. Typically, the inhibitor of GRP-78 is any compound that competes with a GRP-78 ligand for available ligand binding sites on GRP-78. In the context of the invention, the ligand is ISM-1. Accordingly, the inhibitor of GRP-78 prevents the interaction between GRP-78 and ISM-1. GRP-78 inhibitors include compounds that specifically bind to GRP-78, or that can inhibit the activity or expression of GRP-78. Examples include antibodies, small molecule inhibitors, antisense oligonucleotides or siRNA. Typically, the inhibitor of GRP-78 is a small molecule. In a particular embodiment, the small molecule is molecules such as HA15 as described in WO2014072486 and Cerezo et al 2016, Oncoscience, Vil 3(11-12), November 2016. In another embodiment, the small molecule is OSU-03012 (AR-12) which is a celecoxib derivative. OSU-03012 (AR-12) has the CAS number 742112-33-0 and is described in Booth L et al, J Cell Physiol. 2015 July; 230(7):1661-76. doi: 10.1002/jcp.24919; Liu J et al, Anticancer Drugs. 2013 August; 24(7):690-8. doi: 10.1097/CAD.0b013e328362469. In another embodiment, the small molecule is IT-139. IT-139 also called NKP-1339, is developed and commerzialide by Intezyne Technologies Inc. FDA grants orphan drug designation on June 2017 to IT-139 for pancreatic cancer. In a particular embodiment, the small molecule is HKH40 A as described in Kosakowska Cholody et al Cell Death and Disease (2014) 5, e1240; doi:10.1038/cddis.2014.203. In a particular embodiment, the inhibitor of GRP-78 is an antibody. In a particular embodiment, the antibody is a monoclonal antibody. In a particular embodiment, the monoclonal antibody is Mab159 as described in Ren Liu et al Clin Cancer Res. 2013 Dec. 15; 19(24): 6802-6811.

A “therapeutically effective amount” is intended for a minimal amount of active agent which is necessary to impart therapeutic benefit to a subject. For example, a “therapeutically effective amount” to a subject is such an amount which induces, ameliorates or otherwise causes an improvement in the pathological symptoms, disease progression or physiological conditions associated with or resistance to succumbing to a disorder. It will be understood that the total daily usage of the compounds of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidential with the specific compound employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The isthmin-1 inhibitors as described above may be combined with pharmaceutically acceptable excipients, and optionally sustained-release matrices, such as biodegradable polymers, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The polypeptide (or nucleic acid encoding thereof) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

In a particular embodiment, the invention relates to a method of treating INS, MCN or FGFS in a subject in need thereof comprising i) a first step consisting in determining whether the subject suffers from INS, MCN or FGFS according to methods as described above and ii) administering to said subject a therapeutically amount of inhibitor of ISM-1 when the membrane expression level of isthmin-1 or the ratio of the intracellular expression level of isthmin-1 to the membrane expression is higher than its predetermined reference value.

Method and Compositions for Treating Dysfunction of Capillary Permeability

In seventh aspect, the invention relates to a method of treating dysfunction of capillary permeability in a subject in need thereof comprising a step of administering to the subject a therapeutically effective amount of inhibitors of isthmine-1.

In a particular embodiment, the invention relates to a method of treating capillary permeability in a subject in need thereof comprising i) a first step consisting in determining whether the subject suffers from glomerular capillary permeability according to methods as described above and ii) administering to said subject a therapeutically amount of inhibitor of ishtmine-1 when the membrane expression level of isthmin-1 or the ratio of the intracellular expression level of isthmin-1 to the membrane expression is higher than its predetermined reference value.

In a particular embodiment, the method according to the invention is suitable to treat the dysfunction of glomerular capillary permeability in a subject in need thereof.

As used herein, the term “inhibitors of isthmine-1” refers to a natural or synthetic compound that has a biological effect to inhibit or significantly reduce the activity or expression of the transcripts and/or proteins. Such inhibitors are described above.

The invention will be further illustrated by the following figures and examples. However, these examples and figures should not be interpreted in any way as limiting the scope of the present invention.

### FIGURES

FIG. 1: Membrane leucocyte isthmin-1 expression measured by facs in healthy controls (filled round) and in patients with various cause of nephrotic syndrome (empty triangle).

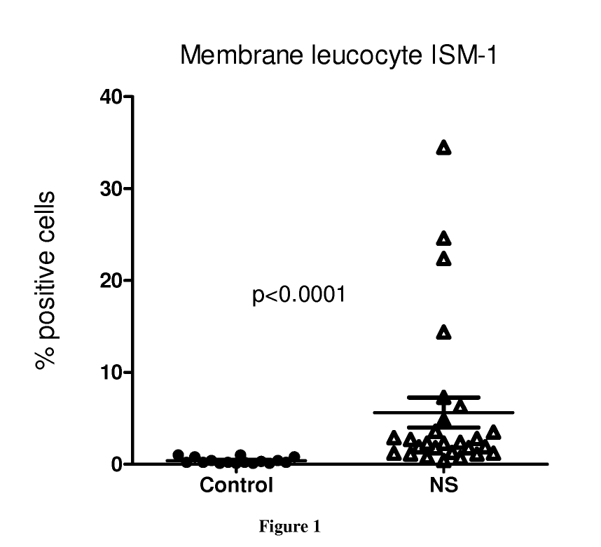

FIG. 2: (A) Membrane leucocyte isthmin-1 expression measured by facs in patients with various causes of nephrotic syndrome. Minimal change disease (MCD) star; secondary MCD (2^{nd} MCD) filled square; primitive FSGS filled round, secondary FSGS filled triangle, membranous nephropathy (MN) empty triangle; Diabetes nephropathy, (empty rounds); amyloidosis, filled triangle; post infectious glomerunephritis, diabolo; lupus glomerunephritis, empty diamond; membranoproliferative glomerulonephritis (GNMP), filled diamond; ANCA vasculitis, Half-filled round. (B) Intracellular leucocyte isthmin-1 expression by facs patients with various causes of nephrotic syndrome. Minimal change disease (MCD) star; secondary MCD (2^{nd} MCD) filled square; membranous nephropathy (MN) empty triangle; Diabetes nephropathy, (empty rounds); amyloidosis, filled triangle; post infectious glomerunephritis, X; lupus glomerunephritis, empty diamond; membranoproliferative glomerulonephritis (GNMP), filled diamond; ANCA vasculitis, Half-filled round.

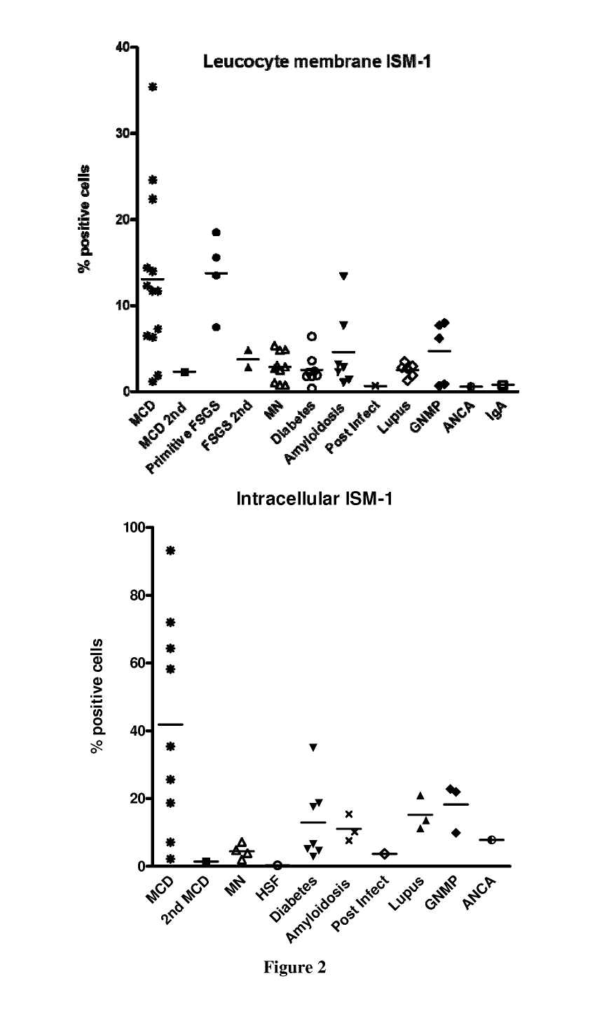

FIGS. 3 and 4: ISM1 inhibition using anti-sens decreases the level of proteiunuria and albuminuria in nephrotic rats. Rats received adriamycin intravenous injection to develop nephrotic syndrome or isotonic saline solution (control group). Urine albumin to creatinine ratio and urine protein to creatinine ratio were measured before, 10, 20 and 30 days after adriamycin injection. Nephrotic rats were treated with either scramble antisens or ISM-1 antisens three time par week intraperitonealy (n=6 per group).

FIG. 5: ISM1 inhibition using anti-sens increases the level of plasma albumin in nephrotic rats. 30 days after adriamycin or saline solution injection, plasma albumin was measured in nephrotic and control rats (n=6 per group).

FIG. 6: Renal function was unchanged in all groups. Plasma creatinine and urea were measured 30 days after adriamycin or saline injection.

FIG. 7: Glomerular ISM-1 and beta5 integrin mRNA expression. 30 days after adriamycine or saline injection, nephritic and control rats were euthanized and kidneys collected for glomeruli isolation. ISM-1 and β5 integrin mRNA expression were measured by RT-PCR (n=6 per group).

FIG. 8: ISM1 inhibition using anti-sens prevents the development of FSGS lesion and synechies. Optic microscopy kidney sections from nephrotic and control rats at 30 days after adriamycin or saline injection were subjected to Masson trichrome staining. Floculus synechies to Bowman capsule and FSGS lesions were quantified in 20 randomly selected glomeruli per section (n=6 rats per group).

FIG. 9: ISM1 inhibition using anti-sens prevents the development of foot process effacement in nephrotic rats. Electronic microscopy section from nephrotic and control rats at 30 days after adriamycin or saline injection were studied. The number of podocyte foot process per length of GBM was calculated on each image performed with the same magnification (n=6 rats per group).

### EXAMPLE

Material & Methods

Population:

Out clinic and hospitalized patients from the nephrology department with the inclusion criterias and consented to participated had a blood sample collection.

Protocol:

Inclusion Criteria:
All adult and children patients with nephrotic syndrome as defined (proteinuria >3 g/j ou>50 mg/kg/m²/d and hypoalbuminemia <30 g/l),
And a histologic diagnosis of MCN or primitive FSGS in adults or a evolutif diagnosis of INS in children.

Non inclusion criteria: nephrotic patients receiving immunosuppressive therapy (corticosteroids and/or immunosuppressor).

Principal outcome: test sensitivity of ISM-1: The diagnosis is made if the expression of ISM-1 circulating leucocyte by flow cytometry at diagnosis of nephrotic syndrome is >20 times the average value of healthy controls.

Secondary Outcomes:
Test specificity, PPV, NPV test in adults.
Sensitivity, Specificity, PPV, NPV test monocyte and granulocyte membrane expression of Miss-1 cytometry

Measurement of ISM-1 Leucocyte Expression by FACS

10 ml EDTA blood sample are collected in patient with inclusion criteria. Samples are quickly addressed to the hematology department for ISM-1 measurement.

Methods

Whole blood sample was collected in an EDTA-containing sterile tube and centrifuged at room temperature at 1500×g for 15 min to separate leukocyte band (buffy coat), plasma and red blood cells pellet (RBCs). After centrifugation, buffy coat was collected, the red blood cells were lysed with KCl solution. The leukocytes were collected after centrifugation at 2500×g for 10 min and washed with PBS. Then cells were fixed in 1% paraformaldehyde during 30 min 4° C., washed twice with PBS and permeabilized with TWEEN 0.1% (5 min, 4° C.).

Cells to be used for flow cytometry analysis were incubated in the dark for 30 min with different antibodies: anti-CD3 (A07746, Beckman Coulter), anti-CD56 (IM2474, Beckman Coulter) anti-CD14 (A07765, Beckman Coulter) anti-CD19 (IM3628, Beckman Coulter) and anti-ISM-1. IgG Isotypes were used for each experiment (mouse IgG1-FITC, A07795; IgG1-APC, IM2475, IgG2A-PC5 and IgG1 PC7 Beckman Coulter). Cells were again washed, re-suspended in PBS and analyzed using flow cytometer FC500 apparatus (Beckman Coulter).

Isthmin-1: A Glomerular Capillary Permeability Factor

To address this question, recombinant isthmin-1 protein (Biolegend) is injected in anesthetized mice. Urine albumin-creatinin ratio is measured before and after ISM-1 injection using a commercial kit (ref).

Isthmin-1 Inhibition Improves Proteinuria

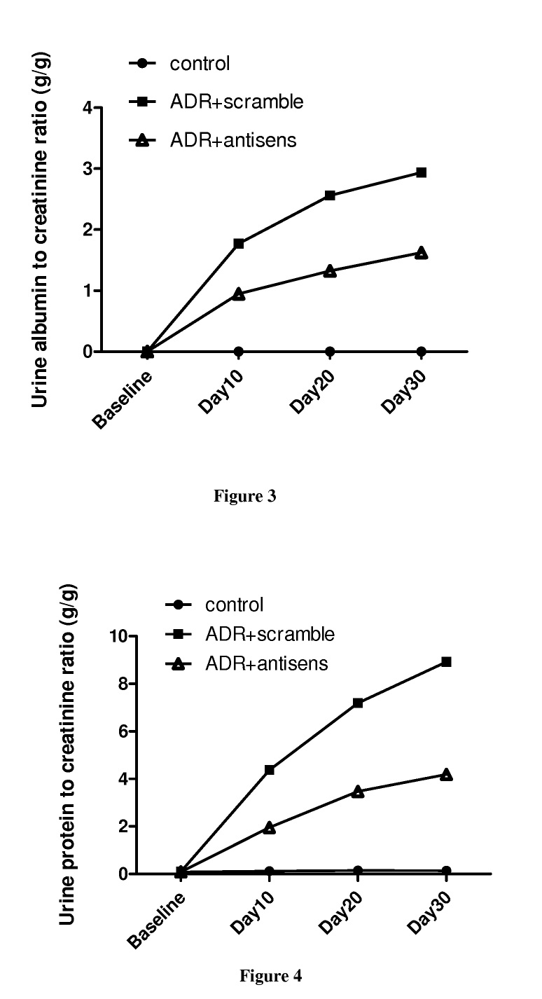

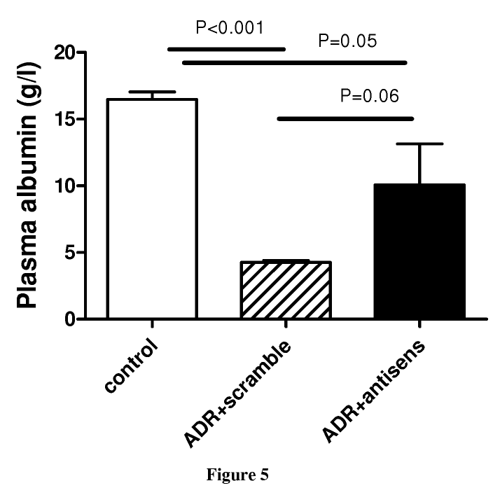

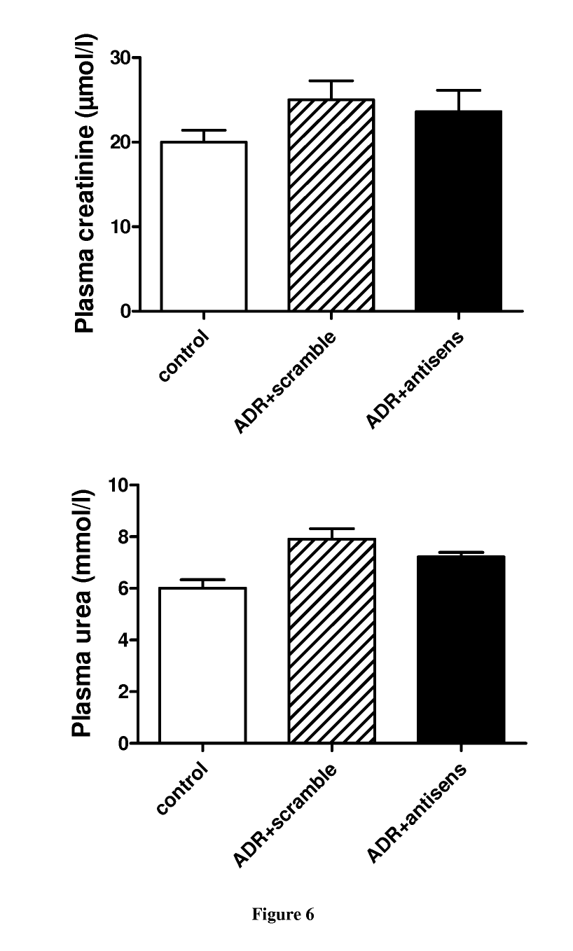

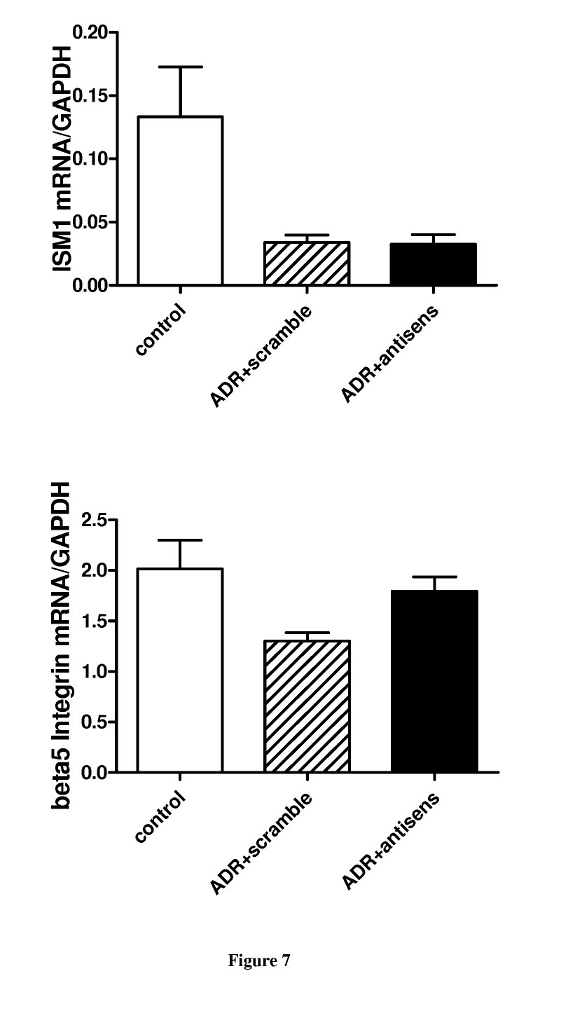

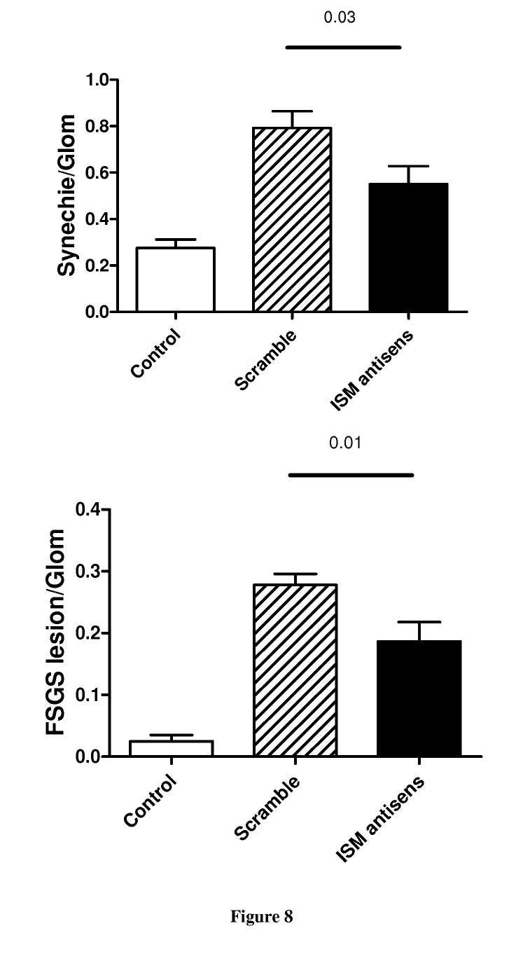

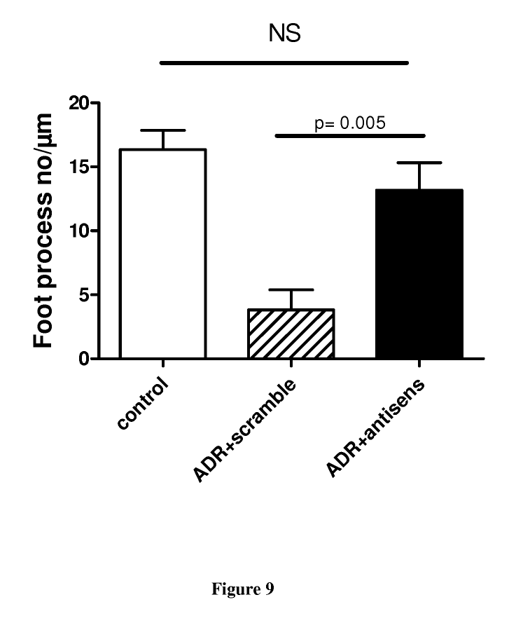

To test the effect of ISM-1 inhibition during nephrotic syndrome, we induced a nephrotic syndrome in rats by adriamycin intravenous injection. These rats develop a heavy proteinuria and hypoalbuminemia with time and were treated with either anti-sens against ISM-1 or scramble anti-sens for 30 days three time per week intraperitonealy. Those groups were compared to a sodium isotonic saline injected group. Measurement of urine albumin and protein to creatinine ratio were measured every 10 days for 30 days and compared between groups. As shown on FIGS. 3 and 4, ISM1 anti-sens significantly decreased the level proteiunuria and albuminuria in nephrotic rats at 30 days after adriamycin injection. Moreover, the FIG. 5 shows that ISM1 anti-sens increased the level of plasma albumin in nephrotic rats. In this model of nephropathy, renal function was unchanged and ISM1 anti-sens had no effect on renal function (FIG. 6). Furthermore, inventors show on FIGS. 8 and 9 that the ISM1 anti-sens prevents the development of FSGS lesions (optic microscopy study) and foot process effacement (electronic microscopy) in nephrotic rats. FIG. 7 suggest that the beneficial effect of ISM-1 inhibition seems independent of glomerular ISM-1 and β5 integrin expression but could be due to an extra-renal effect of ISM1 inhibition.

Accordingly, the results show that the inhibition of ISM1 could prevents and favor recovery of INS, MCN and FSGS, and thus, to treat the nephrotic syndrome.

### REFERENCES

Throughout this application, various references describe the state of the art to which this invention pertains. The disclosures of these references are hereby incorporated by reference into the present disclosure.

1-6. (canceled)

7. The method according to claim 16, wherein the expression level of isthmin-1 is measured by cell sorting (FACS).

8. The method according to claim 16, wherein the expression level of isthmin-1 is measured by ELISA.

9-12. (canceled)

13. The method according to claim 16, wherein the at least one inhibitor of isthmin-1 is an antibody.

14. The method according to claim 16, wherein the at least one inhibitor of isthmin-1 is a small inhibitory RNAs (siRNAs) RNA (siRNA).

15. The method according to claim 16, wherein the inhibitor of isthmin-1 is a small molecule.

16. A method of treating INS, MCN or FSGS in a subject in need thereof comprising
i) measuring the membrane expression level of isthmin-1 on circulating leukocytes in a biological sample obtained from said subject; and
administering least one inhibitor of isthmin-1 to the subject if the membrane expression level of isthmin-1 is higher than a predetermined reference value for the membrane expression level;
or
ii) measuring the intracellular expression level of isthmin-1 in the circulating leukocytes in a sample obtained from said subject;
iii) measuring the membrane expression level of isthmin-1 on the circulating leukocytes in said sample; and
administering least one inhibitor of isthmin-1 to the subject if the ratio of the intracellular expression level of isthmin-1 to the membrane expression level of isthmin-1 is higher than a predetermined reference value for the ratio.

17. A method of treating dysfunction of capillary permeability in a subject in need thereof comprising
measuring the membrane expression level of isthinin-1 on circulating leukocytes in a biological sample obtained from said subject; and
administering at least one inhibitor of isthmin-1 to the subject if the membrane expression level of isthmin-1 is higher than a predetermined reference value for the membrane expression level;
or
measuring the intracellular expression level of isthmin-1 in the circulating leukocytes in a sample obtained from said subject;
measuring the membrane expression level of isthmin-1 on the circulating leukocytes in said sample; and
administering least one inhibitor of isthmin-1 to the subject if the ratio of the intracellular expression level of isthmin-1 to the membrane expression level of isthmin-1 is higher than a predetermined reference value for the ratio.

18. The method according to claim 17, wherein the at least one inhibitor of isthmin-1 is an antibody.

19. The method according to claim 17, wherein the at least one inhibitor of isthmin-1 is a small inhibitory RNA (siRNA).

20. The method according to claim 17, wherein the inhibitor of isthmin-1 is a small molecule.

21. The method according to claim 17, wherein the expression level of isthmin-1 is measured by cell sorting (FACS).

22. The method according to claim 17, wherein the expression level of isthmin-1 is measured by ELISA.

23. An analytic method comprising
obtaining a biological sample comprising circulating leukocytes from a subject having, suspected of having, or being treated for idiopathic nephrotic syndrome (INS), minimal change nephropathy (MCN) or primitive focal segmental glomerulosclerosis (FSGS),
and
measuring the membrane expression level of isthmin-1 on circulating leukocytes in the biological sample.

24. The analytic method of claim 23, further comprising measuring the intracellular expression level of isthmin-1 in the circulating leukocyt
